(19) United States
(12) Patent Application Publication
(10) Pub. No.: US 2006/0223130 A1
(43) Pub. Date: Oct. 5, 2006

(54) Detection of post-translationally modified analytes

(76) Inventors: Dan-Hui Yang, Loveland, CO (US); Magdalena Bynum, Loveland, CO (US)

Correspondence Address: AGILENT TECHNOLOGIES INC.;INTELLECTUAL PROPERTY ADMINISTRATION, LEGAL DEPT,, M/S DU404, P.O. BOX 7599, LOVELAND, CO, 80537-0599, US

(21) Appl. No.: 11/098,912

(22) Filed: Apr. 4, 2005

(51) Int. Cl. G01N 33/53

(52) U.S. Cl. 435/7.92

(57) ABSTRACT

The invention provides methods of analyzing a sample. In general, the methods involve: a) contacting a sample with an array of capture agents; and b) reading the array to detect the presence of a post-translational modification indicator and an analyte-detection moiety. The analytes of the sample may be contacted with the post-translational modification indicator and analyte-detection moiety at any point in the subject methods. Also provided is a system and kit for performing the subject methods.

### BACKGROUND OF THE INVENTION

Post-translational modification of a protein in a cell involves the enzymatic addition of a chemical group, e.g., a phosphate or glycosyl group, to an amino acid of that protein. Such modifications are thought to be required for maintaining and regulating protein structure and function, and abnormal post-translational events have been detected in a wide variety of diseases and conditions, including heart disease, cancer, neurodegenerative and inflammatory diseases and diabetes.

Protein phosphorylation is a type of post-translational modification used to selectively transmit regulatory signals from receptors positioned at the surface of a cell to the nucleus of the cell. The molecules mediating these reactions are predominantly protein kinases that catalyze the addition of phosphate groups onto selected proteins, and protein phosphatases that catalyze the removal of those phosphate groups. Complex biological processes such as cell cycle, cell growth, cell differentiation, and metabolism are orchestrated and tightly controlled by reversible phosphorylation events that modulate protein activity, stability, interactions and localization. Accordingly, protein phosphorylation is thought to play a regulatory role in almost all aspects of cell biology. Perturbations in protein phosphorylation, e.g., by mutations that generate constitutively active or inactive protein kinases and phosphatases, play a prominent role in oncogenesis. Serine, threonine, tyrosine, histidine, arginine, lysine, cysteine, glutamic acid or aspartic acid residues may be phosphorylated. The hydroxyl groups of serine, threonine or tyrosine residues are most commonly phosphorylated.

Protein glycosylation, on the other hand, is acknowledged as being a post-translational modification that has a major effect on protein folding, conformation distribution, stability and activity. Carbohydrates in the form of asparagine-linked (N-linked) or serine/threonine (O-linked) oligosaccharides are major structural components of many cell surface and secreted proteins. All N-linked carbohydrates are linked through N-acetylglucosamine, and most O-linked carbohydrates are attached through N-acetylgalactosamine. O-linked N-acetylglucosamine (O-GlcNAc) is a recently identified type of glycosylation. Unlike classical O- or N-linked protein glycosylation, O-GlcNAc glycosylation involves linking a single GlcNAc moiety to the hydroxyl group of a serine or threonine residue. Increasing evidence suggests that O-GlcNAc modification is a regulatory modification similar to phosphorylation, since it is highly dynamic and rapidly cycles in response to cellular signals.

Because of the central role of post-translational modification in cell biology, much effort has been focused on the development of methods for identifying post-translationally modified proteins. A variety of methods for identifying and characterizing post-translationally modified proteins have been developed.

For example, traditional methods for analyzing phosphorylation sites involve incorporation of radioactive phosphorus into cellular phosphorylated proteins by feeding cells with ³²P ATP. The radioactive proteins can be detected during subsequent fractionation procedures (e.g., two-dimensional gel electrophoresis or high-performance liquid chromatography). Proteins thus identified can be subjected to complete hydrolysis and the phosphoamino acid content determined. The site(s) of phosphorylation can be determined by proteolytic digestion of the radiolabeled protein, separation and detection of phosphorylated peptides (e.g., by two-dimensional peptide mapping), followed by peptide sequencing by Edman degradation. These techniques are generally tedious, require significant quantities of the phosphorylated protein and involve the use of considerable amounts of radioactivity.

In recent years, affinity chromatography has become widely employed in many of methods for identifying post-translational modifications. The most widely used method involves selectively enriching phosphoproteins from a sample using immobilized metal affinity chromatography (IMAC). In this technique, metal ions, usually Fe³⁺ or Ga³⁺, are bound to a chelating support. Phosphoproteins are selectively bound to the column by the affinity of the phosphate moiety of the phosphoproteins to the metal ions of the column. The phosphoproteins can be released using high pH buffer, and subjected to mass spectrometry (MS) analysis. While this method is widely employed, it is limited because many phosphoproteins are unable to bind to IMAC columns, and bound phosphoproteins are often difficult to elute from such columns. Furthermore, these methods produce significant background signals from unphosphorylated proteins that are typically acidic in nature and therefore have affinity for the immobilized metal ions of such columns.

Accordingly, there is an ongoing need for straightforward and reliable methods to identify post-translationally modified proteins in a sample. This invention meets this need, and others.

Publications of interest include: Martin et al, (Proteomics, 2003 3:1244-55); Steinberg et al, (Proteomics, 2003 3:1128-44) and Martin et al, (Comb. Chem. High Throughput Screen., 2003 6:331-9) and published US patent applications US20040180380 and 20050014197.

### SUMMARY OF THE INVENTION

The invention provides a method of analyzing a sample. In general, the method involves: a) contacting a sample with an array of capture agents; and c) reading the array to detect the presence of analytes bound to: i) a post-translational modification indicator and ii) an analyte-detection moiety. The analytes of the sample are contacted with the analyte-detection moiety and the post-translational modification indicator at any point prior to reading the array. The instant method may be employed to evaluate the post-translational modification status of an analyte in two or more samples. Also provided is a system and kit for performing the subject method. The invention finds use in a variety of different medical, research and proteomics applications.

### BRIEF DESCRIPTION OF THE FIGURES

FIG. 1 is a flow diagram describing a first embodiment of the subject invention.

FIG. 2 is a flow diagram describing a second embodiment of the subject invention.

FIG. 3 is a flow diagram describing a third embodiment of the subject invention.

### Definitions

Unless defined otherwise, all technical and scientific terms used herein have the same meaning as commonly understood by one of ordinary skill in the art to which this invention belongs. Still, certain elements are defined below for the sake of clarity and ease of reference.

The term “sample” as used herein relates to a material or mixture of materials, typically, although not necessarily, in fluid form, e.g., aqueous, containing one or more components of interest. Samples may be derived from a variety of sources such as from food stuffs, environmental materials, a biological sample such as tissue or fluid isolated from an individual, including but not limited to, for example, plasma, serum, spinal fluid, semen, lymph fluid, the external sections of the skin, respiratory, intestinal, and genitourinary tracts, tears, saliva, milk, blood cells, tumors, organs, and also samples of in vitro cell culture constituents (including but not limited to conditioned medium resulting from the growth of cells in cell culture medium, putatively virally infected cells, recombinant cells, and cell components).

Components in a sample are termed “analytes” herein. In certain embodiments, the sample is a complex sample containing at least about 10², 5×10³, 10³, 5×10³, 10⁴, 5×10⁴, 10⁵, 5×10⁵, 10⁶, 5×10⁶, 10⁷, 5×10⁷,10⁷, 10⁹, 10¹¹, 10¹¹, 10¹² or more species of analyte.

The term “analyte” is used herein to refer to a known or unknown component of a sample, which will specifically bind to a capture agent on a substrate surface if the analyte and the capture agent are members of a specific binding pair. In general, analytes are biopolymers, i.e., an oligomer or polymer such as an oligonucleotide, a peptide, a polypeptide, an antibody, or the like. In this case, an “analyte” is referenced as a moiety in a mobile phase (e.g., fluid), to be detected by a “capture agent” which, in some embodiments, is bound to a substrate, or in other embodiments, is in solution. However, either of the “analyte” or “capture agent” may be the one which is to be evaluated by the other (thus, either one could be an unknown mixture of analytes, e.g., polypeptides, to be evaluated by binding with the other).

A “biopolymer” is a polymer of one or more types of repeating units, regardless of the source. Biopolymers may be found in biological systems and particularly include polypeptides and polynucleotides, as well as such compounds containing amino acids, nucleotides, or analogs thereof. The term “polynucleotide” refers to a polymer of nucleotides, or analogs thereof, of any length, including oligonucleotides that range from 10⁻¹⁰⁰ nucleotides in length and polynucleotides of greater than 100 nucleotides in length. The term “polypeptide” refers to a polymer of amino acids of any length, including peptides that range from 6-50 amino acids in length and polypeptides that are greater than about 50 amino acids in length.

In most embodiments, the terms “polypeptide” and “protein” are used interchangeably. The term “polypeptide” includes polypeptides in which the conventional backbone has been replaced with non-naturally occurring or synthetic backbones, and peptides in which one or more of the conventional amino acids have been replaced with one or more non-naturally occurring or synthetic amino acids. The term “fusion protein” or grammatical equivalents thereof references a protein composed of a plurality of polypeptide components, that while not attached in their native state, are joined by their respective amino and carboxyl termini through a peptide linkage to form a single continuous polypeptide. Fusion proteins may be a combination of two, three or even four or more different proteins. The term polypeptide includes fusion proteins, including, but not limited to, fusion proteins with a heterologous amino acid sequence, fusions with heterologous and homologous leader sequences, with or without N-terminal methionine residues; immunologically tagged proteins; fusion proteins with detectable fusion partners, e.g., fusion proteins including as a fusion partner a fluorescent protein, β-galactosidase, luciferase, and the like.

In general, polypeptides may be of any length, e.g., greater than 2 amino acids, greater than 4 amino acids, greater than about 10 amino acids, greater than about 20 amino acids, greater than about 50 amino acids, greater than about 100 amino acids, greater than about 300 amino acids, usually up to about 500 or 1000 or more amino acids. “Peptides” are generally greater than 2 amino acids, greater than 4 amino acids, greater than about 10 amino acids, greater than about 20 amino acids, usually up to about 50 amino acids. In some embodiments, peptides are between 5 and 30 amino acids in length.

The term “capture agent” refers to an agent that binds an analyte through an interaction that is sufficient to permit the agent to bind and concentrate the analyte from a homogeneous mixture of different analytes. The binding interaction may be mediated by an affinity region of the capture agent. Representative capture agents include polypeptides and polynucleotides, for example antibodies, peptides or fragments of single stranded or double stranded DNA may employed. Capture agents usually “specifically bind” one or more analytes. For example, antibodies and peptides are types of capture agents.

Accordingly, the term “capture agent” refers to a molecule or a multi-molecular complex which can specifically bind an analyte, e.g., specifically bind an analyte for the capture agent, with a dissociation constant (K_{D}) of less than about 10⁻⁶ M without binding to other targets.

The term “specific binding” refers to the ability of a capture agent to preferentially bind to a particular analyte that is present in a homogeneous mixture of different analytes. In certain embodiments, a specific binding interaction will discriminate between desirable and undesirable analytes in a sample, in some embodiments more than about 10 to 100-fold or more (e.g., more than about 1000- or 10,000-fold). In certain embodiments, the affinity between a capture agent and analyte when they are specifically bound in a capture agent/analyte complex is characterized by a K_{D} (affinity constant) of less than 10⁻⁶ M, less than 10⁻⁷ M, less than 10⁻⁸ M, less than 10⁻⁹ M, usually less than about 10⁻¹⁰ M.

The term “capture agent/analyte complex” is a complex that results from the specific binding of a capture agent with an analyte, i.e., a “binding partner pair”. A capture agent and an analyte for the capture agent specifically bind to each other under “conditions suitable for specific binding”, where such conditions are those conditions (in terms of salt concentration, pH, detergent, protein concentration, temperature, etc.) which allow for binding to occur between capture agents and analytes to bind in solution. Such conditions, particularly with respect to antibodies and their antigens, are well known in the art (see, e.g., Harlow and Lane (Antibodies: A Laboratory Manual Cold Spring Harbor Laboratory, Cold Spring Harbor, N.Y. (1989)). Conditions suitable for specific binding typically permit capture agents and target pairs that have a dissociation constant (K_{D}) of less than about 10⁻⁶ M to bind to each other, but not with other capture agents or targets.

As used herein, “binding partners” and equivalents refer to pairs of molecules that can be found in a capture agent/analyte complex, i.e., exhibit specific binding with each other.

The phrase “surface-bound capture agent” refers to a capture agent that is immobilized on a surface of a solid substrate, where the substrate can have a variety of configurations, e.g., a sheet, bead, or other structure, such as a plate with wells. In certain embodiments, the collections of capture agents employed herein are present on a surface of the same support, e.g., in the form of an array.

The term “pre-determined” refers to an element whose identity is known prior to its use. For example, a “pre-determined analyte” is an analyte whose identity is known prior to any binding to a capture agent. An element may be known by name, sequence, molecular weight, its function, or any other attribute or identifier. In some embodiments, the term “analyte of interest”, i.e., an known analyte that is of interest, is used synonymously with the term “pre-determined analyte”.

The terms “antibody” and “immunoglobulin” are used interchangeably herein to refer to a capture agent that has at least an epitope binding domain of an antibody. These terms are well understood by those in the field, and refer to a protein containing one or more polypeptides that specifically binds an antigen. One form of antibody constitutes the basic structural unit of an antibody. This form is a tetramer and consists of two identical pairs of antibody chains, each pair having one light and one heavy chain. In each pair, the light and heavy chain variable regions are together responsible for binding to an antigen, and the constant regions are responsible for the antibody effector functions.

The recognized immunoglobulin polypeptides include the kappa and lambda light chains and the alpha, gamma (IgG₁, IgG₂, IgG₃, IgG₄), delta, epsilon and mu heavy chains or equivalents in other species. Full-length immunoglobulin “light chains” (of about 25 kDa or about 214 amino acids) comprise a variable region of about 110 amino acids at the NH₂-terminus and a kappa or lambda constant region at the COOH-terminus. Full-length immunoglobulin “heavy chains” (of about 50 kDa or about 446 amino acids), similarly comprise a variable region (of about 116 amino acids) and one of the aforementioned heavy chain constant regions, e.g., gamma (of about 330 amino acids).

The terms “antibodies” and “immunoglobulin” include antibodies or immunoglobulins of any isotype, fragments of antibodies which retain specific binding to antigen, including, but not limited to, Fab, Fv, scFv, and Fd fragments, chimeric antibodies, humanized antibodies, single-chain antibodies, and fusion proteins comprising an antigen-binding portion of an antibody and a non-antibody protein. The antibodies may be detectably labeled, e.g., with a radioisotope, an enzyme which generates a detectable product, a fluorescent protein, and the like. The antibodies may be further conjugated to other moieties, such as members of specific binding pairs, e.g., biotin (member of biotin-avidin specific binding pair), and the like. The antibodies may also be bound to a solid support, including, but not limited to, polystyrene plates or beads, and the like. Also encompassed by the terms are Fab′, Fv, F(ab′)₂, and or other antibody fragments that retain specific binding to antigen.

Antibodies may exist in a variety of other forms including, for example, Fv, Fab, and (Fab′)₂, as well as bi-functional (i.e. bi-specific) hybrid antibodies (e.g., Lanzavecchia et al., Eur. J. Immunol. 17, 105 (1987)) and in single chains (e.g., Huston et al., Proc. Natl. Acad. Sci. U.S.A., 85, 5879-5883 (1988) and Bird et al., Science, 242, 423-426 (1988), which are incorporated herein by reference). (See, generally, Hood et al., “Immunology”, Benjamin, N.Y., 2nd ed. (1984), and Hunkapiller and Hood, Nature, 323, 15-16 (1986)). Monoclonal antibodies and “phage display” antibodies are well known in the art and encompassed by the term “antibodies”.

The term “mixture”, as used herein, refers to a combination of elements, e.g., capture agents or analytes, that are interspersed and not in any particular order. A mixture is homogeneous and not spatially separable into its different constituents. Examples of mixtures of elements include a number of different elements that are dissolved in the same aqueous solution, or a number of different elements attached to a solid support at random or in no particular order in which the different elements are not specially distinct. In other words, a mixture is not spatially addressable. To be specific, a spatially addressable array of capture agents, as is commonly known in the art and described in greater detail below, is not a mixture of capture agents because the species of capture agents are spatially distinct and the array is addressable.

“Isolated” or “purified” generally refers to isolation of a substance (compound, polynucleotide, protein, polypeptide, polypeptide composition) such that the substance comprises a significant percent (e.g., greater than 2%, greater than 5%, greater than 10%, greater than 20%, greater than 50%, or more, usually up to about 90%-100%) of the sample in which it resides. In certain embodiments, a substantially purified component comprises at least 50%, 80%-85%, or 90-95% of the sample. Techniques for purifying polynucleotides and polypeptides of interest are well-known in the art and include, for example, ion-exchange chromatography, affinity chromatography and sedimentation according to density. Generally, a substance is purified when it exists in a sample in an amount, relative to other components of the sample, that is not found naturally.

The term “assessing” includes any form of measurement, and includes determining if an element is present or not. The terms “determining”, “measuring”, “evaluating”, “assessing” and “assaying” are used interchangeably and may include quantitative and/or qualitative determinations. Assessing may be relative or absolute. “Assessing the presence of” includes determining the amount of something present, and/or determining whether it is present or absent.

The term “array” encompasses the term “microarray” and refers to an array of capture agents for binding to aqueous analytes and the like.

An “array,” includes any two-dimensional or substantially two-dimensional (as well as a three-dimensional) arrangement of spatially addressable regions (i.e., “features”) containing capture agents, particularly antibodies, and the like. Where the arrays are arrays of proteinaceous capture agents, the capture agents may be adsorbed, physisorbed, chemisorbed, or covalently attached to the arrays at any point or points along the amino acid chain. In some embodiments, the capture agents are not bound to the array, but are present in a solution that is deposited into or on features of the array.

Any given substrate may carry one, two, four or more arrays disposed on a surface of the substrate. Depending upon the use, any or all of the arrays may be the same or different from one another and each may contain multiple spots or features. A typical array may contain one or more, including more than two, more than ten, more than one hundred, more than one thousand, more ten thousand features, or even more than one hundred thousand features, in an area of less than 20 cm² or even less than 10 cm², e.g., less than about 5 cm², including less than about 1 cm², less than about 1 mm², e.g., 100 μm², or even smaller. For example, features may have widths (that is, diameter, for a round spot) in the range from a 10 μm to 1.0 cm. In other embodiments each feature may have a width in the range of 1.0 μm to 1.0 mm, usually 5.0 μm to 500 μm, and more usually 10 μm to 200 μm. Non-round features may have area ranges equivalent to that of circular features with the foregoing width (diameter) ranges. At least some, or all, of the features are of the same or different compositions (for example, when any repeats of each feature composition are excluded the remaining features may account for at least 5%, 10%, 20%, 50%, 95%, 99% or 100% of the total number of features). Inter-feature areas will typically (but not essentially) be present which do not carry any proteins or nucleic acids (or other biopolymer or chemical moiety of a type of which the features are composed). Such inter-feature areas typically will be present where the arrays are formed by processes involving drop deposition of reagents but may not be present when, for example, photolithographic array fabrication processes are used. It will be appreciated though, that the inter-feature areas, when present, could be of various sizes and configurations. The term “array” encompasses the term “microarray” and refers to any one-dimensional, two-dimensional or substantially two-dimensional (as well as a three-dimensional) arrangement of spatially addressable regions, usually bearing biopolymeric capture agents, e.g., polypeptides, nucleic acids, and the like.

Each array may cover an area of less than 200 cm², or even less than 50 cm² 5 cm², 1 cm², 0.5 cm², or 0.1 cm². In certain embodiments, the substrate carrying the one or more arrays will be shaped generally as a rectangular solid (although other shapes are possible), having a length of more than 4 mm and less than 150 mm, usually more than 4 mm and less than 80 mm, more usually less than 20 mm; a width of more than 4 mm and less than 150 mm, usually less than 80 mm and more usually less than 20 mm; and a thickness of more than 0.01 mm and less than 5.0 mm, usually more than 0.1 mm and less than 2 mm and more usually more than 0.2 and less than 1.5 mm, such as more than about 0.8 mm and less than about 1.2 mm.

Arrays can be fabricated using drop deposition from pulse-jets of either precursor units (such as nucleotide or amino acid monomers) in the case of in situ fabrication, or the previously obtained capture agent.

An array may be spatially addressable or optically addressable. An array is “spatially addressable” when it has multiple regions of different moieties (e.g., different capture agents) such that a region (i.e., a “feature” or “spot” of the array) at a particular predetermined location (i.e., an “address”) on the array will detect a particular sequence. Array features are typically, but need not be, separated by intervening spaces. An “optically addressable” array contains an aqueous population of capture agents that are labeled with a optically distinguishable tags. The individual species of capture agent of an optically addressable array are usually bound to the same solid substrate (e.g., a bead or plurality thereof) and are linked to an optically detectable tag (e.g., a fluorophore) so that they can be separated and distinguished from other capture agents. Optically addressable arrays of capture agents readily adaptable to the instant methods are described in greater detail in U.S. Pat. Nos. 6,649,414 and 6,524,793.

An “array layout” refers to one or more characteristics of the features, such as feature positioning on the substrate, one or more feature dimensions, and an indication of a moiety at a given location.

The term “post-translationally modification indicator”, as will be described in greater detail below, is any molecule that can bind to and indicate the presence of a post-translationally modified analyte. For example, post-translationally modification indicator may bind to and indicate the presence of a phosphoproteins (i.e., a protein that has been phosphorylated) or a glycoprotein (i.e. a protein that has been glycosylated). Post-translational modification indicators do not detectably bind to analytes that are not post-translationally modified.

The term “analyte detection moiety”, as will be described in greater detail below, is any molecule that can indicate the presence of an analyte, regardless of the post-translational modification status of the analyte. In other words, an analyte detection moiety binds to both the post-translationally modified and non-post-translationally modified forms of an analyte.

If a first element is “bound to” a second element, the binding between those elements may be either direct or indirect (e.g., by means of third element that simultaneously binds to both the first and the second elements). The linkage between a first element bound to a second element may be covalent or non-covalent.

The term “using” has its conventional meaning, and, as such, means employing, e.g., putting into service, a method or composition to attain an end. For example, if a program is used to create a file, a program is executed to make a file, the file usually being the output of the program. In another example, if a computer file is used, it is usually accessed, read, and the information stored in the file employed to attain an end. Similarly if a unique identifier, e.g., a barcode is used, the unique identifier is usually read to identify, for example, an object or file associated with the unique identifier.

### DETAILED DESCRIPTION OF THE INVENTION

The invention provides a method of analyzing a sample. In general, the method involves: a) contacting a sample with an array of capture agents; and c) reading the array to detect the presence of analytes bound to: i) a post-translational modification indicator and ii) an analyte-detection moiety. The analytes of the sample are contacted with the analyte-detection moiety and the post-translational modification indicator at any point prior to reading the array. The instant method may be employed to evaluate the post-translational modification status of an analyte in two or more samples. Also provided is a system and kit for performing the subject method. The invention finds use in a variety of different medical, research and proteomics applications.

Before the present invention is described in such detail, however, it is to be understood that this invention is not limited to particular variations set forth and may, of course, vary. Various changes may be made to the invention described and equivalents may be substituted without departing from the true spirit and scope of the invention. In addition, many modifications may be made to adapt a particular situation, material, composition of matter, process, process act(s) or step(s), to the objective(s), spirit or scope of the present invention. All such modifications are intended to be within the scope of the claims made herein.

Methods recited herein may be carried out in any order of the recited events which is logically possible, as well as the recited order of events. Furthermore, where a range of values is provided, it is understood that every intervening value, between the upper and lower limit of that range and any other stated or intervening value in that stated range is encompassed within the invention. Also, it is contemplated that any optional feature of the inventive variations described may be set forth and claimed independently, or in combination with any one or more of the features described herein.

The referenced items are provided solely for their disclosure prior to the filing date of the present application. Nothing herein is to be construed as an admission that the present invention is not entitled to antedate such material by virtue of prior invention.

Reference to a singular item includes the possibility that there are plural of the same items present. More specifically, as used herein and in the appended claims, the singular forms “a,” “an,” “said” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

In further describing the subject invention, the subject methods are described first, followed by a description of a system for analyzing a sample in which the subject methods may be employed. Kits for use in performing the subject methods will then be described.

Methods for Sample Analysis

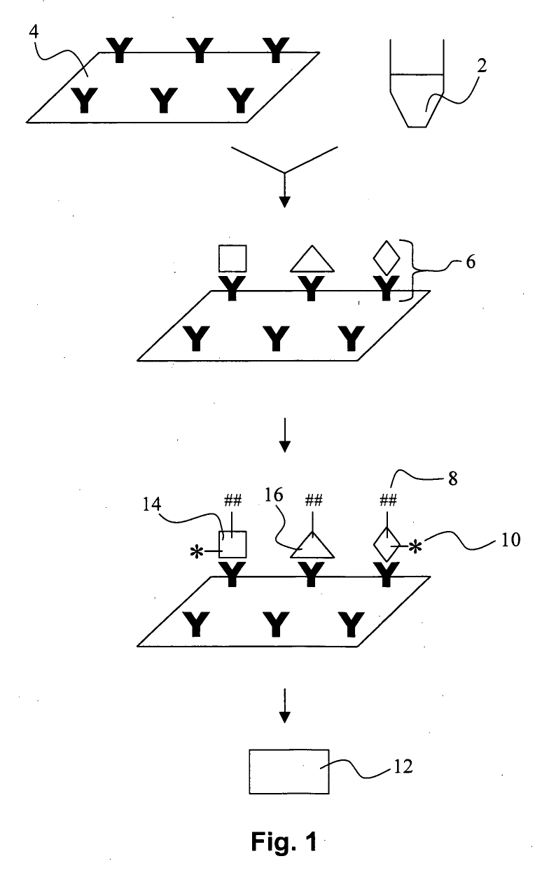

The instant methods may be generally described with reference to the embodiment schematically illustrated in FIG. 1. In one embodiment and with reference to FIG. 1, the instant method involves contacting a sample 2 with an array of capture agents 4 under conditions suitable for specific binding of analytes in the sample to the arrayed capture agents to form capture agent/analyte complexes 6. The array is then read to detect both: a) an analyte-bound analyte detection moiety 8, and b) an analyte-bound post-translational modification indicator 10, to produce data 12.

Also included in the subject methods are steps in which the analytes of the sample are labeled with i) an analyte detection moiety, and ii) a post-translational modification indicator. As will be discussed in greater detail below, the analytes may be labeled with an analyte detection moiety and/or post-translational modification indicator at any point prior to reading the array in the above-recited method. For example, and as will be described in greater detail below, the analytes of the sample may be labeled with an analyte detection moiety and/or post-translational modification indicator prior to contact of the sample with the array, or after contact of the sample with the array. The analyte detection moiety and post-translational modification indicator need not be contacted with the analytes at the same point in the subject method. For example, the sample may be contacted with the analyte detection moiety prior to contacting the sample with the array, whereas the array may be contacted with the post-translational modification indicator to label analytes bound thereto.

Detection of analyte detection moiety 8 at a particular feature indicates the presence of a particular bound analyte. Detection of a post-translational modification indicator 10 at the same feature indicates that the analyte is post-translationally modified. An evaluation of the post-translational modification status (e.g., the phosphorylation or glycosylation status) of a particular analyte may be provided by comparing the amount of an analyte detection moiety at a particular feature to the amount of a post-translational modification indicator at the same feature. In the exemplary embodiment shown in FIG. 1, the method discriminates between post-translationally modified analyte 14 and non-post-translationally modified analyte 16, and allows the post-translational modification status of an analyte (e.g., how much of a particular analyte is phosphorylated or glycosylated) to be determined. As will be described in greater detail below, the subject methods may also be used to identify analytes having a post-translational modification status that is different in different samples (i.e., to identify analytes that are differentially post-translationally modified in different samples), and to investigate how the post-translational modification status of a particular analyte may change in response to a stimulus, for example.

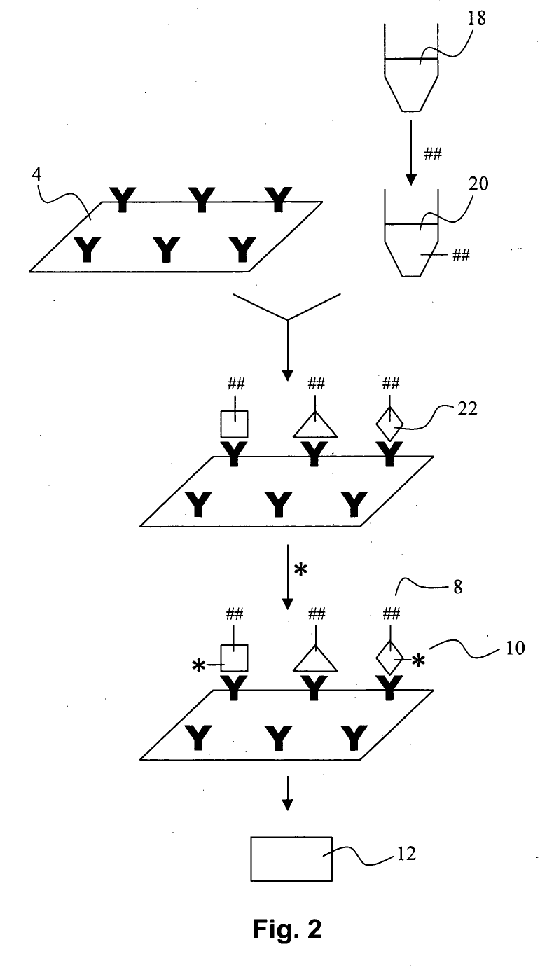

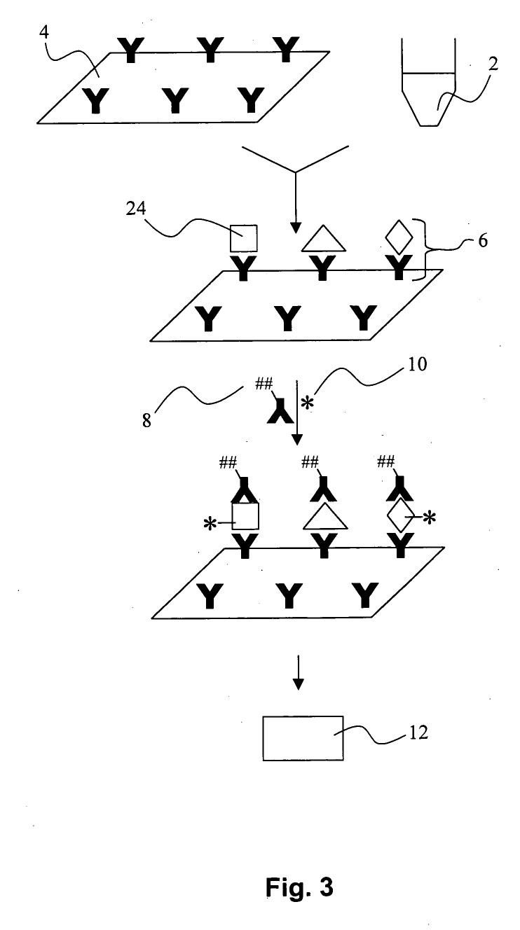

FIG. 2 and FIG. 3 illustrate further embodiments of the invention. In a first embodiment of the invention and as illustrated in FIG. 2, unlabeled sample 18 is labeled with an analyte detection moiety to produce labeled sample 20, which is then contacted with capture agent array 4 to produce capture agent/analyte complexes containing analyte detection moiety-labeled analyte 22. Post-translationally modified analytes that are bound to a capture agent, e.g., bound analyte 14, are labeled by contacting the array with a post-translational modification indicator (*). The array is then read to detect both: a) analyte-bound analyte detection moiety 8, and b) analyte-bound post-translational modification indicator 10, to produce data 12. In other words, a labeled sample is made by labeling an unlabeled sample with an analyte detection moiety, and the labeled sample is contacted with a capture agent array. After this step, the array is contacted with a post-translational modification indicator, and analytes that are post-translationally modified are identified by reading the array to detect features that are labeled by both the analyte detection moiety and post-translational modification indicator.

In a second embodiment of the invention and as illustrated in FIG. 3, sample 2 is contacted with capture agent array 4 to produce capture agent/analyte complexes 6. Post-translationally modified analytes that are bound to a capture agent, e.g., bound analyte 24, are labeled by contacting the array with analyte detection moiety 8 and post-translational modification indicator 10, simultaneously, or in any order. The array is then read to detect both: a) an analyte-bound analyte detection moiety, and b) an analyte-bound post-translational modification indicator, to produce data 12. In other words, a capture agent array is first contacted with an unlabeled sample, and after this step the array is contacted with an analyte-bound analyte detection moiety and an analyte-bound post-translational modification indicator (in any order or simultaneously). The array is then read to identify analytes that are post-translationally modified.

In the embodiments shown in FIG. 3, analyte detection moiety 8 is a labeled second capture agent for a particular analyte, where the first capture agent for that analyte is bound to the array substrate. Analyte detection systems containing first and second capture agents for a particular analyte (sometimes known as “sandwich” assay systems) are generally well known in the analyte detection arts (see, e.g., Sambrook, et al., Molecular Cloning: A Laboratory Manual, Second Edition, (1989) Cold Spring Harbor, N.Y.).

In describing these methods in greater detail, post-translational modification indicators and analyte detection moieties will be described first, followed by a discussion of how those elements may be employed in combination with a capture agent array to evaluate the presence of post-translationally modified analytes.

Post-Translational Modification Indicators and Analyte Detection Moieties

Post-translational modification indicators are detectable moieties that specifically bind to analytes, e.g., polypeptides, that are post-translationally modified. A variety of post-translational modification indicators may be employed in the subject methods. In particular embodiments a labeled antibody or post-translational modification-specific dye may be used. For example, to identify phosphoproteins (i.e., polypeptides to which a phosphate group has been added), any one or more of a variety of labeled anti-phosphotyrosine, anti-phosphoserine or anti-phosphothreonine antibodies may be used. Such antibodies may be purchased from a variety of different manufacturers, including Research Diagnostics Inc. (Flanders N.J.), Zymed Laboratories, Inc. (San Francisco, Calif.), PerkinElmer (Torrance, Calif.) and Sigma-Aldrich (St. Louis, Mo.). Alternatively, dyes (particularly fluorescent dyes) that specifically bind to phosphoproteins may be employed. Such dyes include methyl green (Cutting et al, Analytical Biochemistry 1973 54, 386-394) sold by Pierce (Rockford, Ill.), among others, and the phosphopeptide-specific PRO-Q DIAMOND™ dye of Molecular Probes (Eugene, Oreg.). Further details of a wide variety of suitable phosphoprotein-specific dyes, in particular metal chelating moiety-containing phosphoproteins-specific dyes, are found in U.S. Published Patent Applications US20040171034, US20040038306 and US20050014197, which applications is incorporated herein in their entirety. Such dyes typically bind to any amino acid containing a phosphate group, including phosphoserine, phosphotyrosine and phosphothreonine.

Likewise, to identify glycoproteins, one or more of a variety of anti-glycoprotein antibodies may be employed (see product literature of Novus (Littleton, Colo.) and Sigma-Aldrich (St. Louis, Mo.), for example). A variety of glyco-specific dyes, e.g., SYPRO RUBY™, PRO-Q FUCSIA™ and PRO-Q EMERALD™ dyes, e.g., PRO-Q EMERALD 300™ or PRO-Q EMERALD 488™, of Molecular Probes (Eugene, Oreg.) may also be employed. Other dyes suitable for employment in the instant methods are disclosed in U.S. Ser. No. 09/970,215 (published as US20020137068).

Analyte detection moieties, unlike post-translational modification indicators, are detectable moieties that bind to an analyte and thereby indicate the presence of the analyte, regardless of whether the analyte is post-translationally modified (e.g., phosphorylated or glycosylated). In other words, an analyte detection moiety binds to the non-modified and modified forms of an analyte, typically with the same or similar affinities. An analyte detection moiety may be analyte specific (e.g., may be an analyte-specific antibody) or non-specific (e.g., may be a general polypeptide labeling reagent, e.g., a polypeptide-specific dye or detectable conjugant or the like).

Post-translational modification indicators and analyte detection moieties generally contain a directly or indirectly detectable label, where a directly detectable labels is a label that provides a directly detectable signal without interaction with one or more additional chemical agents. Examples of directly detectable labels include fluorescent labels. Indirectly detectable labels are those labels which interact with one or more additional members to provide a detectable signal. In this latter embodiment, the label may be a member of a signal producing system that includes two or more chemical agents that work together to provide the detectable signal. Examples of indirectly detectable labels include biotin, streptavidin or digoxigenin, which can be detected by a binding partner (e.g., an antibody or the like) coupled to a fluorochrome, for example.

In use, the labels of a post-translational modification indicator and an analyte detection moiety may be distinguishable, meaning that the post-translational modification indicator and the analyte detection moiety can be independently detected and measured, even when they are mixed. In other words, the amounts of post-translational modification indicator and analyte detection moiety present (e.g., the amount of fluorescence) are separately determinable, even when the molecules are co-located (e.g., in the same tube or in the same duplex molecule or in the same feature of an array). Suitable distinguishable fluorescent label pairs useful in the subject methods include Cy-3 and Cy-5 (Amersham Inc., Piscataway, N.J.), Quasar 570 and Quasar 670 (Biosearch Technology, Novato Calif.), Alexafluor555 and Alexafluor647 (Molecular Probes, Eugene, Oreg.), BODIPY V-1002 and BODIPY V1005 (Molecular Probes, Eugene, Oreg.), POPO-3 and TOTO-3 (Molecular Probes, Eugene, Oreg.), and POPRO3 and TOPRO3 (Molecular Probes, Eugene, Oreg.). Further suitable distinguishable detectable labels may be found in Kricka et al. (Ann Clin Biochem. 39:114-29, 2002). In particular embodiments in which a fluorescent dye that specifically bind to a post-translationally modified analyte is used, the analyte detection moiety may be chosen so that it has a label that is distinguishable from the fluorescent dye. For example, if the phosphopeptide-specific dye PRO-Q DIAMOND™ is employed as a post-translational modification indicator, that dye may be used in conjunction with an analyte detection moiety that is labeled with cyanine-5 (Cy-5).

Methods of labeling analytes samples for use in array-based experiments are generally well known in the art and are described in, for example, Zhu et al (Science, 2001 293: 2101-2105), Huang et al (Proc. Natl. Acad. Sci., 2004 101:16594-9), Saviranta et al (Clin. Chem., 2004 50:1907-20), Ge et al (Nucleic Acids Res., 2000 28:e3); Lin et al, (Cancer Lett. 2002 187:17-24), Anderson et al, (Brain 2003 126:2052-64) and Ivanof et al, (Mol. Cell Proteomics, 2004 3:788-95). These art-known methods are readily adapted to the instant methods. Methods of using post-translational modification-specific dyes such as PRO-Q DIAMOND™ to detect post-translationally modified analytes in an array format are described in the product literature supplied with those dyes, as well as Martin et al, (Proteomics, 2003 3:1244-55), Steinberg et al, (Proteomics, 2003 3:1128-44) and Martin et al (Comb. Chem. High Throughput Screen., 2003 6:331-9), and need not be described herein in any more detail.

Methods of Sample Analysis

The subject invention employs an array of capture agents. Such an array may generally comprise a plurality of spatially addressable features (e.g., more than about 10, more than about 100, more than about 500, more than 1000, features, usually up to about 110,000 or more features) that contain capture agents. In certain embodiments, a single species of capture agent is present in each of the features, however, in other embodiments, a feature may contain a mixture of different capture agents.

In certain embodiments of the invention, the capture agents are proteinaceous capture agents, methods for the making of which are generally well known in the art. For example, polypeptides may be produced in bacterial, insect or mammalian cells (see, e.g., Ausubel, et al., Short Protocols in Molecular Biology, 3rd ed., Wiley & Sons 1995 and Sambrook et al., Molecular Cloning: A Laboratory Manual, Third Edition, 2001 Cold Spring Harbor, N.Y.) using recombinant means, isolated, and deposited onto a suitable substrate.

Capture agents may be selected based on their binding to pre-determined analytes in a sample. Accordingly, in the subject methods, the pre-determined analytes and the capture agents that bind those analytes are selected prior to starting the subject methods. In other embodiments, the capture agents are not pre-determined and their binding specificity may be unknown.

Capture agents may be chosen using any means possible. For example, sets of capture agents present on an array may bind to proteins of a particular signal transduction, developmental or biochemical pathway, proteins having similar biological functions, proteins of similar size or structure, or they may bind proteins that are known markers for a biological condition or disease. Capture agents may also be chosen at random, or on the availability of capture agents, e.g., if a capture agent is available for purchase, for example. In some embodiments, a capture agent may be chosen purely because it is desirable to know whether the analyte to which the capture agent binds is post-translationally modified. The binding partner for a capture agent does not have to be known for the capture agent to be present on an array for use in the subject methods.

In certain embodiments, a single capture agent will bind to a single analyte. Accordingly, a set, i.e., a plurality, of capture agents for analysis is chosen. In certain embodiments, each of these capture agents binds to a single species of analyte, and does not discriminate between non-post-translationally modified and post-translationally modified forms of the analyte. In other words, since an array of capture agents usually contains more than about 4, more than about 8, more than about 12, more than about 24, more than about 48, more than about 96, more than about 192, or more than about 384 or more features containing different capture agents, a corresponding number of different analytes may be present or may be suspected of being present in the sample to be assessed. In certain embodiments, there are about 50-500 different capture agents on a subject array.

Further, since the capture agents are chosen using any means possible, there is no requirement that any or all of the analytes for those capture agents are present in a sample to be analyzed. In fact, since the subject methods may be used to determine the presence or absence of an analyte in a sample, as well as the post-translational modification status of the analyte in a sample, only a fraction or none of the analytes may be present in a sample to be analyzed.

In particular embodiments, capture agents are monoclonal antibodies, although any molecule that can specifically bind an analyte, e.g., other types of proteins, such as members of known binding partner pairs, antibodies such as phage display antibodies and antibody fragments or the like, may be used. Monoclonal antibodies that specifically bind to analytes are well known in the art and may be made using conventional technologies (see, e.g., Harlow and Lane, Antibodies: A Laboratory Manual Cold Spring Harbor Laboratory, Cold Spring Harbor, N.Y. (1989)). Monoclonal antibodies that specifically bind to known analytes may also be purchased from a number of antibody suppliers such as Santa Cruz Biotechnology, Santa Cruz, Calif. and Epitomics, Inc., Burlingame, Calif. Antibody fragments and phage display antibodies are also well known in the art.

In certain embodiments, it is desirable to employ a capture agent that is free of post-translation modifications to avoid any undesirable molecular interactions with a post-translational modification indicator. Accordingly, non-post-translationally modified capture agents may be employed in certain embodiments. Such capture agents may be produced using a variety of means. For example, synthetic capture agents (e.g., peptides) can be employed. Antibodies that are produced in mammalian cells are not phosphorylated and are therefore suitable for use in methods that employ a phosphopeptide indicator. Antibodies can be produced in a bacterial cell to avoid glycosylation, or, in alternative embodiments, phage display antibodies or antibody fragments that contain no glycosylation sites may be employed in the instant methods.

Methods for making arrays of polypeptides using contact and inkjet (i.e., piezoelectric) deposition methods are generally well known in the art (see e.g., U.S. Pat. Nos. 6,372,483, 6,352,842, 6,346,416 and 6,242,266; MacBeath and Schreiber, Science (2000) 289:1760-3). Specific methods for producing polypeptide arrays are also found in Zhu et al (Science, 2001 293: 2101-2105); Huang et al (Proc. Natl. Acad. Sci., 2004 101:16594-9); Saviranta et al (Clin. Chem., 2004 50:1907-20); Ge et al (Nucleic Acids Res., 2000 28:e3); Lin et al, (Cancer Lett. 2002 187:17-24); Anderson et al, (Brain 2003 126:2052-64) and Ivanof et al, (Mol. Cell Proteomics, 2004 3:788-95).

In one embodiment of the instant sample analysis method, a proteinaceous sample is directly labeled (e.g., with a fluorescent label such as Cy-5) or indirectly labeled (e.g., with, for example, biotin) and the labeled sample is contacted with a capture agent array under conditions suitable for specific binding of the analytes of the sample to the capture agents of the array. The array is then contacted with a post-translational modification indicator that specifically binds only to analytes that are post-translationally modified.

In a second embodiment of the instant sample analysis method, an unlabeled proteinaceous sample is contacted with a capture agent array under conditions suitable for specific binding of the analytes of the sample to the capture agents of the array. The array is then contacted with a) a post-translational modification indicator that specifically binds only to analytes that are post-translationally modified and b) directly or indirectly labeled second capture agents that bind to capture agent-bound analytes regardless of whether they are post-translationally modified. Methods similar to those described by Saviranta (Clin. Chem. 2004 50:1907-20) are readily employed in this embodiment.

In a particular embodiment, the instant methods employ array staining methods similar to those described by Martin et al, (Proteomics, 2003 3:1244-55) to identify post-translationally modified analytes.

The array is read using an array reader (e.g., an array scanner). Details of scanners and scanning procedures that may be employed in the subject methods are found in U.S. Pat. Nos. 6,806,460, 6,791,690 and 6,770,892, for example.

Numerical data corresponding to the amount of post-translational modification indicator and analyte detection moiety associated with features of the array are produced using feature extraction software. In a representative embodiment, two sets of data are produced, each set corresponding to a distinguishable label employed in the assay. Each numerical value associated with an amount of signal for a feature may also be associated with other numerical values pertaining to the quality of the label signal from the feature (e.g., values associated with the variation in pixel intensity for a feature), as is commonly provided by the commercially available programs. Amounts of signal may be measured as a quantitative (e.g., absolute) value of signal, or a qualitative (e.g., relative) value of signal, as is known in the art.

In certain embodiments of the instant methods, the data obtained from reading a subject array is processed to express the signal for the post-translational modification indicator for a feature relative to the signal of the analyte detection moiety for that feature. The signal of the post-translational modification indicator indicates the amount of a particular post-translationally modified analyte bound by the feature, and the signal of the analyte detection moiety indicates the total amount of the analyte bound by the feature, regardless of whether that feature is modified or not modified. This expression may be in the form of a ratio (e.g., a fraction, any integer, a number, or any log thereof), and indicates the post-translational modification status of the analyte.

For example, if an analyte detection moiety is detected at a particular feature, the presence or absence of a post-translational modification indicator at that feature indicates that the analyte bound at that feature is either post-translationally modified or not post-translationally modified, respectively. The relative level of the post-translational modification indicator as compared to the analyte detection moiety indicates the post-translational modification status of the analyte.

In accordance with the above, the invention further provides a system for sample analysis. In general, the subject system contains: a) an array of capture agents, b) a post-translational modification indicator, and c) an analyte detection moiety (e.g., a set of second capture agents), details of which are described in greater detail above. The system may also contain an array reader, and any hardware or other reagents necessary to perform the above methods.

Utility

The subject methods may be employed in a variety of diagnostic, drug discovery, and research applications that include, but are not limited to, diagnosis or monitoring of a disease or condition (where the degree of post-translational modification of a particular analyte is a marker for the disease or condition), discovery of drug targets (where the analyte is differentially post-translationally modified in a disease or condition and may be targeted for drug therapy), drug screening (where the effects of a drug are monitored by assessing the level of post-translational modification of an analyte), determining drug susceptibility (where drug susceptibility is associated with a particular profile of post-translational modifications) and basic research (where it is desirable to identify the presence of a post-translationally modified analyte in a sample, or, in certain embodiments, the relative levels of a post-translationally modified analyte in two or more samples).

In particular embodiments, the instant methods may be used to identify post-translationally modified polypeptides, including polypeptides that have been phosphorylated or glycosylated. In these embodiments, a sample is analyzed using the above methods, and the identity of some or all of the post-translationally modified polypeptides in the sample can be determined. In certain embodiments, the subject methods may be employed to produce a “profile” of post-translationally modified polypeptides for a sample.

In certain embodiments, a sample may be analyzed to determine if a particular post-translationally modified polypeptide is present in the sample.

In other embodiments, relative post-translational modification status of an analyte of two or more different samples may be obtained using the above methods, and compared. In these embodiments, the results obtained from the above-described methods are usually normalized to the total amount of analyte present (as indicated by the analyte detection moiety), and compared. This may be done by comparing ratios, as described above, or by any other means. In particular embodiments, the post-translational modification profiles of two or more different samples may be compared to identify post-translational modification events that are associated with a particular disease or condition (e.g., a phosphorylation or glycosylation event that is induced by the disease or condition and therefore may be part of a signal transduction pathway implicated in that disease or condition).

The different samples may consist of an “experimental” sample, i.e., a sample of interest, and a “control” sample to which the experimental sample may be compared. In many embodiments, the different samples are pairs of cell types or fractions thereof, one cell type being a cell type of interest, e.g., an abnormal cell, and the other a control, e.g., normal, cell type. If two fractions of cells are compared, the fractions are usually the same fraction from each of the two cells. In certain embodiments, however, two fractions of the same cell may be compared. Exemplary cell type pairs include, for example, cells isolated from a tissue biopsy (e.g., from a tissue having a disease such as colon, breast, prostate, lung, skin cancer, or infected with a pathogen etc.) and normal cells from the same tissue, usually from the same patient; cells grown in tissue culture that are immortal (e.g., cells with a proliferative mutation or an immortalizing transgene), infected with a pathogen, or treated (e.g., with environmental or chemical agents such as peptides, hormones, altered temperature, growth condition, physical stress, cellular transformation, etc.), and a normal cell (e.g., a cell that is otherwise identical to the experimental cell except that it is not immortal, infected, or treated, etc.); a cell isolated from a mammal with a cancer, a disease, a geriatric mammal, or a mammal exposed to a condition, and a cell from a mammal of the same species, preferably from the same family, that is healthy or young; and differentiated cells and non-differentiated cells from the same mammal (e.g., one cell being the progenitor of the other in a mammal, for example). In one embodiment, cells of different types, e.g., neuronal and non-neuronal cells, or cells of different status (e.g., before and after a stimulus on the cells) may be employed. In another embodiment of the invention, the experimental material is cells susceptible to infection by a pathogen such as a virus, e.g., human immunodeficiency virus (HIV), etc., and the control material is cells resistant to infection by the pathogen. In another embodiment of the invention, the sample pair is represented by undifferentiated cells, e.g., stem cells, and differentiated cells. The subject methods are particularly employable in methods of detecting the phosphorylation status of phosphorylated serum proteins.

Accordingly, among other things, the instant methods may be used to link certain post-translational modifications (i.e., a certain modification of a certain protein) to certain physiological events.

In particular embodiments, the subject methods may be used to establish cellular signaling pathways that are employed to transmit signals in a cell (e.g., from the exterior or interior of the cell to a cell nucleus, or from one protein in a cell to another, directly or indirectly). For example, the subject methods may be employed to determine the phosphorylation status of a protein in a cell (e.g., determine how much of a particular protein is phosphorylated at any moment in time), thereby indicating the activity of the kinase or phosphates for which that protein is a substrate, even if the identity of the kinase or phosphates is unknown. The substrates for a particular kinase or phosphatase may be identified by virtue of the fact that they should be phosphorylated or dephosphorylated by the same stimulus, at the same point in time. A signal transduction pathway for a particular stimulus may be determined by identifying all of the phosphorylation/dephosphorylation events for a particular stimulus, and determining when those events occur. Certain post-translational modifications that occur before other post-translational modifications (e.g., immediately after a stimulus) are generally upstream in a signal transduction pathway, whereas other post-translational modifications that occur after other post-translational modifications (e.g., long after a stimulus) are generally at the end of a signal transduction pathway.

In one embodiment, the invention provides a method of screening for an agent that modulates post-translational modification. The method generally comprises contacting a candidate agent with a sample and assessing the sample according to the above-recited methods. In certain embodiments, the results from this assay may be compared to those of a sample that has not been contacted with the candidate agent. Such a method may be employed to identify an agent that reduces or increases the abundance of a particular post-translationally modified analyte.

A variety of different candidate agents may be screened by the above methods. Candidate agents encompass numerous chemical classes, though typically they are organic molecules, preferably small organic compounds having a molecular weight of more than 50 and less than about 5000 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Agents that modulate post-translational modification typically decrease or increase the amount of a post-translationally modified analyte (relative to the total amount of that analyte) by at least about 10%, at least about 20%, at least about 50%, at least about 70%, or at least about 90%.

Kits

Also provided by the subject invention are kits for practicing the subject methods, as described above. The subject kits contain at least a post-translational modification indicator and an analyte detection moiety, as described above. The kit may also contain an array of capture agents. The various components of the kit may be present in separate containers or certain compatible components may be precombined into a single container, as desired.

In addition to above-mentioned components, the subject kits may further include instructions for using the components of the kit to practice the subject methods, i.e., to instructions for sample analysis. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g., CD-ROM, diskette, etc. In yet other embodiments, the actual instructions are not present in the kit, but means for obtaining the instructions from a remote source, e.g., via the internet, are provided. An example of this embodiment is a kit that includes a web address where the instructions can be viewed and/or from which the instructions can be downloaded. As with the instructions, this means for obtaining the instructions is recorded on a suitable substrate.

In addition to the subject database, programming and instructions, the kits may also include one or more control analyte mixtures, e.g., two or more control samples for use in testing the kit.

### EXAMPLES

The following examples are put forth so as to provide those of ordinary skill in the art with a complete disclosure and description of how to make and use the present invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

A mixture of cellular or plasma proteins is diluted in binding buffer. The antibody microarrays are then incubated with the solution at room temperature overnight. The arrays are washed twice in 200 mL of phosphate buffer (pH 7.2) containing 0.05% tween-20 for 10 min, and then washed once in phosphate buffer (pH 7.2) for 10 min. The arrays are rinsed briefly with water before spin-drying in a centrifuge.

The arrays are then incubated with the mixture Cy5-labeled secondary antibodies. The secondary antibodies have a concentration of about 300-500 ng/mL for each antibody. The antibodies have specific binding with target proteins regardless their post-translational modifications. The binding process lasts for two hours at room temperature. The arrays are washed twice in 200 mL of phosphate buffer (pH 7.2) containing 0.05% tween-20 for 10 min, and then washed once in phosphate buffer (pH 7.2) for 10 min. The arrays are rinsed briefly with water before spin-drying in a centrifuge.

The above arrays are incubated with Pro-Q Diamond stain solution (Molecular Probes, Inc. P33300) at room temperature in dark. After washing off unused stain solution, the arrays are scanned with fluorescence scanner at dual laser wavelength for Cy3 and Cy5 (Agilent Technologies, Inc., G2565BA). The target concentration and phosphorylation status are calculated based on the standard solution reading.

### Example 2

The mixture of cellular proteins or plasma proteins is directly labeled with Cy5 as described by the Pat Brown et al (Genome Biology, 2001, Vol 2, No. 2). After removal of unlabeled free dye, the labeled protein solution is diluted with the binding buffer. The antibody microarrays are then incubated with the solution at room temperature overnight. The arrays are washed twice in 200 mL of phosphate buffer (pH 7.2) containing 0.05% tween-20 for 10 min, and then washed once in phosphate buffer (pH 7.2) for 10 min. The arrays are rinsed briefly with water before spin-drying in a centrifuge.

The above arrays are incubated with Pro-Q Diamond stain solution (Molecular Probes, Inc. P33300) at room temperature in dark. After washing off unused stain solution, the arrays are scanned with fluorescence scanner at dual laser wavelength for Cy3 and Cy5 (Agilent Technologies, Inc., G2565BA). The target concentration and phosphorylation status are calculated based on the standard solution reading.

The above results and discussion demonstrate a new method for analyzing a sample to identify post-translationally modified analytes in the sample. The instant methods are superior to currently used methods because the instant methods are capable of assessing every analyte in a sample for binding to an agent, and are not limited to the use of metal ion columns. Further, since array technology is readily employed in the instant methods, the instant method is significantly cheaper and higher throughput that prior methods. Accordingly, as such, the subject methods represent a significant contribution to the art.

All publications and patents cited in this specification are herein incorporated by reference as if each individual publication or patent were specifically and individually indicated to be incorporated by reference. The citation of any publication is for its disclosure prior to the filing date and should not be construed as an admission that the present invention is not entitled to antedate such publication by virtue of prior invention.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1. A method of sample analysis, the method comprising:
(a) contacting a sample with an array of capture agents;
(b) contacting said array with a post-translational modification-specific fluorescent dye and an analyte-specific analyte detection moiety; and
(c) reading said array to independently detect:
i) a said post-translational modification-specific fluorescent dye; and
ii) said analyte-specific analyte detection moiety.

2-3. (canceled)

4. The method of claim 1, wherein said capture agents are antibodies.

5. The method of claim 1, wherein said capture agents are peptides.

6-8. (canceled)

9. The method of claim 1, wherein said post-translational modification fluorescent dye specifically binds phospho-amino acids or glycosyl-amino acids.

10. The method of claim 9, wherein said fluorescent dye specifically binds phosoho-amino acids.

11. The method of claim 9, wherein said fluorescent dye specifically binds glycosyl-amino acids.

12. The method of claim 1, wherein said analyte-detection moiety comprises an antibody.

13. The method of claim 1, wherein said analyte-detection moiety comprises a fluorescent tag.

14. The method of claim 1, wherein said post-translational modification-specific fluorescent dye and said analyte-specific analyte detection moiety are distinguishably labeled.

15. A method of sample comparison, the method comprising:
analyzing a first sample by the method of claim 01 to produce sample 1 data;
analyzing a second sample by the method of claim 1 to produce sample 2 data; and
comparing said sample 1 data and said sample 2 data.

16. The method of claim 15, wherein said method indicates an analyte that is differentially post-translationally modified in said first and second samples.

17. The method of claim 15, wherein said comparing step provides a qualitative or quantitative evaluation.

18. A system for sample analysis, comprising:
a post-translational modification indicator;
an analyte detection moiety; and
an array of capture agents.

19. A kit comprising:
a post-translational modification indicator; and
an analyte detection moiety.

20. The kit of claim 19, wherein said analyte detection moiety comprises an antibody.

21. The kit of claim 19, wherein said post-translational modification indicator comprises a compound that specifically binds to phospho-amino acids or glycosyl-amino acids.

22. The kit of claim 19, further comprising an array of capture agents.

23. The kit of claim 19, further comprising instructions to perform the method of claim 1.

24. A method of screening for an agent that modulates post-translational modification, the method comprising:
contacting a candidate agent with a sample; and
assessing said sample according to the method of claim 1.

25. The method of claim 1, wherein said capture agents are selected based on their ability to bind a predetermined set of analytes in a sample.

26. The method of claim 25, wherein said capture agents bind to proteins that are known markers for a biological condition or a disease.

27. The method of claim 1, wherein said capture agents bind to proteins of a signal transduction pathway.
